(19) United States
(12) Patent Application Publication
(10) Pub. No.: US 2021/0100470 A1
(43) Pub. Date: Apr. 8, 2021

(54) ELECTROENCEPHALOGRAPHY (EGG) SENSING ASSEMBLY

(71) Applicant: NEUROSERVO INC., Boucherville (CA)

(72) Inventor: Nicolas Tremblay, Boucherville (CA)

(21) Appl. No.: 17/125,392

(22) Filed: Dec. 17, 2020

Related U.S. Application Data
(63) Continuation of application No. 15/663,306, filed on Jul. 28, 2017.
(60) Provisional application No. 62/368,396, filed on Jul. 29, 2016.

(51) Int. Cl. A61B 5/0482; A61B 5/04; A61B 5/00

(52) U.S. Cl. CPC A61B 5/0482; A61B 5/04001; A61B 5/04012; A61B 5/7445; A61B 5/6814; A61B 5/04017; A61B 5/6803

(57) ABSTRACT

There is provided an electroencephalogram (EEG) sensing assembly. The EEG sensing assembly includes a support layer having an outer side and an opposite inner side for facing a head of a user and at least one sensing electrode. The at least one sensing electrode includes an electrically conductive layer positioned against the inner side of the support layer, a resilient member positioned between the support layer and the electrically conductive layer, and a pair of cooperating snap rings including a first snap ring positioned against the conductive layer and including prongs for engaging through the electrically conductive layer, the resilient member, and the support layer and a second snap ring positioned against the outer side of the support layer and including a socket for snap-fitting the prongs of the first snap ring for retaining together the electrically conductive layer, the resilient member and the support layer.

### CROSS-REFERENCE

The present application is a continuation of U.S. patent application Ser. No. 15/663,306 entitled “NEUROFEEDBACK HEADGEAR FOR MONITORING BRAIN ACTIVITY” filed on Jul. 28, 2017 and which claims priority on U.S. Provisional Patent Application No. 62/368,396 filed on Jul. 29, 2016.

### TECHNICAL FIELD

The technical field generally relates to devices for monitoring brain activity.

### BACKGROUND

Electroencephalography (EEG) involves the monitoring of brain activity through the measuring of electrophysiological signals of the brain. A typical EEG sensor includes one or more electrodes placed along the scalp. The electrodes capture voltage fluctuations resulting from ionic current within the neurons of the brain.

EEG equipment has been used extensively for medical and research purposes.

However, portable EEG applications have been limited.

### SUMMARY

According to one aspect of the present invention, a neurofeedback headgear is provided. The neurofeedback headgear comprises a head receiving portion, a brim portion, an EEG sensor assembly and an emitter. The head receiving portion has an outer side and an inner side, the inner side contacting the head of the user when worn. The inner side comprises a concealing layer. The brim portion extends from the head receiving portion, and has an upper side and an under side. The EEG sensor assembly comprises at least one sensing electrode located on the inner side of the head receiving portion, for contacting the forehead of the user and sensing brain activity when the headgear is worn. The EEG sensor assembly also comprises a microcontroller in communication with the sensing electrode(s). The microcontroller is mounted to the inner side of the head receiving portion and concealed under the concealing layer. The microcontroller analyzes the brain activity sensed by the sensing electrode(s) to determine a state of brain activity of the user. The emitter is located on the underside of the brim portion of the headgear and is in signal communication with the microcontroller. The emitter emits a visual feedback according to the state of brain activity determined by the microcontroller, the visual feedback being located within a field of vision of the user when the headgear is worn by the user.

According to another aspect, a method is provided for sensing/detecting brain activity. The method includes detecting brain activity of a user using at least one electroencephalogram (EEG) sensor assembly mounted onto a neurofeedback headgear being worn by the user and operating an emitter in response to the brain activity detected by the EEG sensor assembly. The wearable emitter is preferably a light emitting device, providing a visual feedback viewable inside a field of vision of the user when the wearable light emitting device is worn by said user.

According to yet another aspect, a wearable electrode is provided. The electrode includes a conductive layer, a resilient backing member supporting the conductive layer, and cooperating snap rings retaining the conductive layer and the resilient backing member, and being attachable to a wearable article.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1 is a schematic diagram of the operational modules of a neurofeedback headgear according to one example embodiment;

FIG. 2A is a perspective view of the underside of a neurofeedback headgear, according to one example embodiment;

FIG. 2B is a perspective view of the underside of a baseball cap according to a configuration well known in the art;

FIG. 3 is a perspective view of a PCB module adapted for stitching, according to one example embodiment;

FIG. 4 is an exploded view of a sensing electrode according to one example embodiment;

FIG. 5A is a close-up view of an assembled sensing electrode attached to the inner sweat band of a headgear, according to one example embodiment;

FIG. 5B shows the rear side of the sensing electrode, connected to a PCB module;

FIG. 6 is a cross-sectional view of a sensing electrode, according to one possible embodiment;

FIG. 7 is a flowchart of the operative steps of a method for sensing brain activity according to one example embodiment; and

FIG. 8 is a schematic diagram of the operational modules of a neurofeedback headgear, according to an alternative example embodiment.

### DETAILED DESCRIPTION

In the following description, the same numerical references refer to similar elements. The embodiments, geometrical configurations, materials mentioned and/or dimensions shown in the figures or described in the present description are embodiments only, given solely for exemplification purposes.

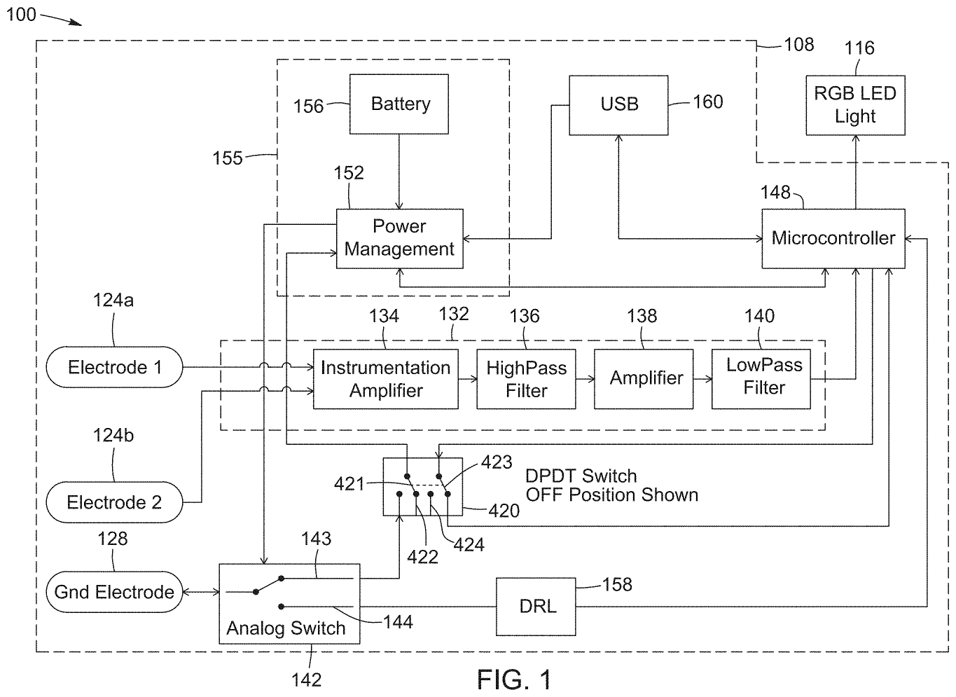

Referring now to FIG. 1, therein illustrated is a schematic diagram of an assembly 100 of operational modules of a neurofeedback headgear according to one example embodiment. The neurofeedback headgear, which may also be referred to as a wearable device, can be operated as a portable EEG system to monitor brain activity of a user wearing the neurofeedback headgear. The operational modules of the neurofeedback headgear include an EEG sensor assembly 108 and at least one wearable emitter 116. The emitter 116 may be worn by a user and may be controlled to provide sensory feedback to the user. In the illustrated example, the wearable emitter is a wearable light emitting device 116.

The EEG sensor assembly 108 is operable to receive and process EEG signals that represent the brain activity of a human user. As illustrated in FIG. 1, the EEG sensor assembly 108 includes one or more sensing electrodes 124 a, 124 b. The sensing electrodes 124 a, 124 b may be any electrode that can capture electrophysiological signals that represent brain activity of a human user. For example, a sensing electrode may include a conductive layer for contacting the scalp or forehead of a human user. The EEG sensor assembly 108 may further include a ground electrode 128 that acts as a reference for the sensing electrodes 124 a, 124 b.

The EEG sensor assembly 108 may further include a signal conditioning module 132 that receives the electrophysiological signals captured by the one or more sensing electrodes 124 a, 124 b. Each sensing electrode 124 a, 124 b may be in electrical signal communication with the signal receiving/conditioning module 132 such that electrophysiological signals captured by a sensing electrode are received at the signal receiving module 132. It will be understood that while FIG. 1 illustrates a single signal conditioning module 132 according to one example embodiment, in other example embodiments the EEG sensor assembly 108 may include a plurality of signal conditioning modules 132 each configured to receive electrophysiological signals captured by one or more sensing electrodes connected to that conditioning module.

According to one example embodiment, and as illustrated, the signal conditioning module 132 includes an instrument amplifier 134, a high-pass filter 136, a second amplifier 138 and a low-pass filter 140. These amplifiers/filters of the signal receiving/conditioning module 132 interoperate to condition/treat the raw electrophysiological signals captured by the one or more sensing electrodes 124 a, 124 b to output a treated EEG signal that is ready for further processing or analysis.

Continuing with FIG. 1, the EEG sensor assembly 108 further includes a microcontroller 148 which receives the EEG signals captured by the one or more sensing electrodes 124 a, 124 b and treated by the signal conditioning module 132. The microcontroller 148 analyses the received EEG signals and determines a state of brain activity of the user based on the received EEG signals. Different algorithms adapted to analyse EEG signals and determine brain activity, including a concentration level of the user and/or other information, can be stored in and executed by the microcontroller.

The EEG sensor assembly 108 further includes a power management module 155 that includes a power management circuit 152 and a battery 156. The battery 156 supplies power to of various elements and operational modules of the wearable device 100. The power supply is managed by the power management circuit 152.

The EEG sensor assembly 108 may further include an input/output port 160. The input/output port 160 allows the neurofeedback headgear to be connected to an external device. When connected, data pertaining to detected brain signals calculated by the microcontroller 148 may be transmitted via the input/output port 160 to the external device. Furthermore, the input/output port 160 may also be operated to receive a source of power to charge the battery 156. For example, and as illustrated, the input/output port 160 is a USB port. The input/output port may also be used to update the firmware of the microcontroller.

The power management module 155 manages the supply of power to various elements of the neurofeedback headgear. When the EEG sensor assembly 108 is connected to an external source of power, such as via the input/output port 160, the power management circuit 152 may supply power from the external source to various elements of the neurofeedback headgear while also charging the battery 156. When the external source of power is not available, the power management circuit 152 may supply power from the battery 156 to the various elements of the neurofeedback headgear.

As described elsewhere herein, the power management module 155 participates in the automatic switching on of the neurofeedback headgear upon the neurofeedback headgear being worn by a user to begin sensing of brain activity. Upon the neurofeedback headgear being worn, the power management module 155 supplies power to the microcontroller 148 and other elements of the neurofeedback headgear to automatically begin sensing of brain activity.

The neurofeedback headgear may optionally include a switch 142, which may be an analog switch that is operable to permit passage of analog signals. The switch 142 may be connected with one of the electrodes 124 a, 124 b or 128. The switch 142 is configured to be toggled in response to detecting contact of the skin of the wearer with at least one electrode, which corresponds to the neurofeedback headgear being worn.

As illustrated, the switch 142 is in a detecting position (ex: pole connected to upper throw 143) in which it provides a signal path between the ground electrode 128 and the power management circuit 152. Upon the neurofeedback headgear being worn by the user, a change in voltage between one of the sensing electrodes 124 a, 124 b and the electrode 128 occurs. This may be detected by the power management module 152 as a drop in voltage at the ground electrode 128. In response to detecting this change, the power management circuit 152 toggles the switch 142 to a sensing position (ex: switch connected to lower throw 144) so that the ground electrode 128 is connected to the microcontroller 148. The power management circuit 152 may further send an actuation signal to the microcontroller 148 to begin the sensing of brain activity.

In one example embodiment, the power management circuit 152 may first send an actuation signal to the microcontroller 148 to inform the microcontroller 148 that the neurofeedback headgear is being worn. The microcontroller 148 may selectively return a signal to the power management circuit 152 based on a current operating state or current configuration of the microcontroller 148. The microcontroller 148 may be in a state or configuration that allows it to begin sensing in response to the neurofeedback headgear being worn, in which case the return signal may indicate to the power management circuit 152 to toggle the switch 142. Alternatively, the microcontroller 148 may be in a state or configuration that does not permit it to begin sensing, which case the return signal indicates that the power management circuit 152 should not toggle the switch 142 or that the no return signal is sent.

It will be appreciated that prior to the neurofeedback headgear being worn, the neurofeedback headgear is in an idle state in which power is received only at the power management circuit 152 and the switch 142 is in a position to allow the power management circuit 152 to detect contact of the electrode 124 a, 124 b, 128 with the skin of the user.

Still referring to FIG. 1, the EEG sensing subassembly 108 may further include an interrupter module 420 that allows selecting whether the microcontroller 148 can be automatically operated to begin sensing brain activity upon initial contact of the electrodes 124 a, 124 b, 128 with the skin of the user when putting on the headgear. Accordingly, the interrupter module 420 allows the neurofeedback headgear to be operated in an “automatic on” mode (mode in which the power management module 152 and the switch 142 detect the neurofeedback headgear 100 being worn by the user) and an “off” mode (mode in which wearing the neurofeedback headgear is not detected). According to one example embodiment, and as illustrated, the interrupter module 420 is a double pole double throw switch. The switch may be physically provided within the neurofeedback headgear, such as on/off switch 396, and may be toggled by the wearer between its two positions.

A first position of the switch of the interrupter module 420 corresponds to the “off mode” and is illustrated in FIG. 1. In this position, a first pole 421 of the switch is toggled to be connected to an open-ended throw 422. Accordingly, the ground electrode 128 is disconnected from the power management module 152 and change in voltage between the electrodes corresponding to the wearer putting on the neurofeedback headgear is not detected at the power management module 152. Furthermore, in this position, a second pole 423 of the switch is toggled to form a connection from the microcontroller 148 back to itself (ex: a short connection). This allows the microcontroller 148 to detect that the interrupter module 420 is in its “off mode” and that the microcontroller 148 should cease and/or not begin sensing of brain activity.

Upon the position of the switch of the interrupter module 420 being toggled to its second position corresponding to the “automatically mode”, the first pole 421 of the switch is toggled to form a connection between the ground electrode 128 and the power management circuit 152 so that contact of electrodes with the skin of the wearer is detected. The second pole 423 of the switch is toggled so that microcontroller 148 is connected to an second open-ended throw 424, to indicate that the microcontroller 148 can be automatically turned on to begin sensing of brain activity.

When the microcontroller 148 is operating to sense brain activity, the further toggling of the switch back to the first position causes the second pole 423 to form the connection of the microcontroller 148 back to itself, indicating that the sensing of brain activity should be stopped. This may correspond to the user toggling the switch of the interrupter module 420 to manually turn off the neurofeedback headgear.

Continuing with FIG. 1, the one or more emitters 116 is/are preferably operable to emit a visually perceptible signal. The emitter 116 may be implemented using any technology known in the art for emitting light, such as a light emitting diode (LED). The emitter 116 may output a plurality of different visually perceptible signals, such as light signals of different colors and/or durations. For example, the emitter 116 may be an RGB LED. While the present embodiment describes the wearable emitter as being a light emitting device, other types of wearable emitters that provide a sensory feedback to the user are possible, such as a sound emitter or a vibrating module. The wearable emitter can be any type of emitter that can alert or indicate to the user of the neurofeedback headgear that a given type of brain activity has been detected by the EEG sensor assembly 108.

The emitter 116 is in signal communication with the EEG sensor assembly 108, and more specifically with the microcontroller 148. The microcontroller 148 is operable to emit a plurality of control signals for controlling the emission of the visually perceptible signals from the light emitting device 116. The control signals transmitted by the microcontroller 148 are based on the analysis of the received sensed brain signals so that different visual signals being emitted by the light emitting device 116 indicate different states of brain activity of the user.

Still referring to FIG. 1, the EEG sensor assembly preferably includes a Driven Right Leg (DRL) circuit 158 in series with the analog switch 142 and the microcontroller 148. The DRL circuit 158 allows reducing common-mode interference produced by the body of the neurofeedback headgear.

According to a possible embodiment, the neurofeedback headgear is a cap or any hat with a head receiving portion and a brim portion extending therefrom. Within the neurofeedback headgear, at least the EEG sensor assembly 108 is physically mounted to the headgear, on the inner side, such that it is concealed. According to one example embodiment, the emitter 116 is also mounted on the headgear.

According to other example embodiments, the emitter 116 is implemented on another wearable article that is separate from the headgear article. Accordingly, the light emitting device 116 is in wireless signal communication with the microcontroller 148 of the EEG sensor assembly 108. The light emitting device 116 may be mounted to a separate wearable article that will allow the light emitting device 116 to be located within the field of vision of the human user that is wearing the headgear article. For example, the light emitting device 116 may be mounted onto a wearable bracelet or within an eyewear article.

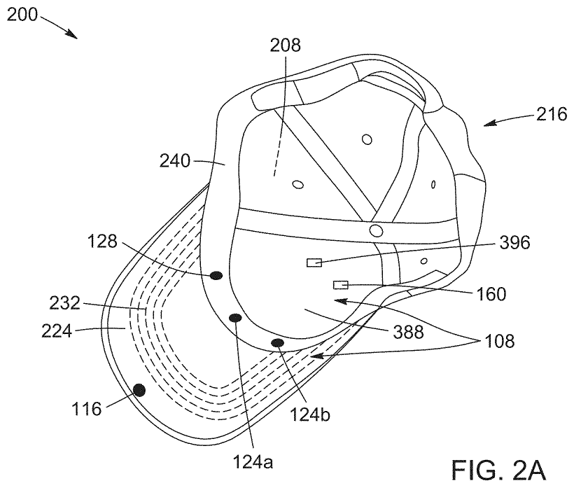

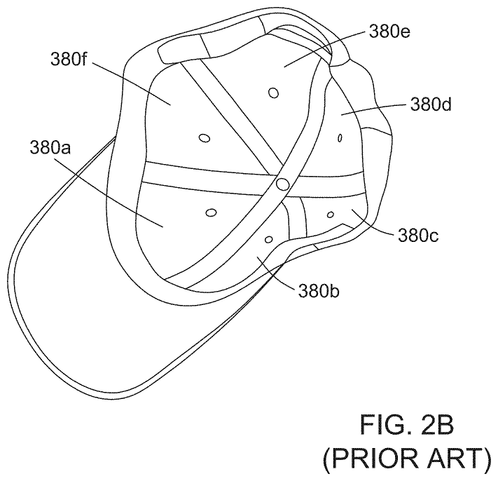

Referring now to FIG. 2A, therein illustrated is a perspective view of the underside of a neurofeedback headgear 200 according to one example embodiment onto which the operational modules discussed previously, including the EEG sensor assembly 108, have been physically mounted. Physical mounting of the EEG sensor assembly 108 to the headgear article 200 herein refers to a mutual arrangement such that when neurofeedback headgear 200 is worn over the head of user, the components of the EEG sensor assembly 108 are also worn on the person's head.

According to one example embodiment, the components of the EEG sensor assembly 108 are physically mounted to the neurofeedback headgear 200 such that they are concealed from view when the headgear article 200 is worn on the head of the human user. In one example embodiment, at least some of the components of the EEG sensor assembly 108 are disposed on an interior surface 208 of the headgear article 200. The interior surface 208 refers to the surface of the headgear article 200 that faces the skin or hear of the person when the headgear article 200 is worn properly.

The neurofeedback headgear 200 may further include an inner concealing layer 388 that is disposed over at least some of the components of the sensor assembly 108, such as the microcontroller. Preferably, the power management module 155, the signal conditioning module 132 and the DRL circuit 158 are also concealed. Accordingly, these components are sandwiched between the interior surface 208 and the inner concealing layer 388 such that they are concealed from view even when viewing the interior of the headgear article 200. However, at least the conductive portion of the one or more sensing electrodes 124 a, 124 b of the EEG sensor system 100 are exposed so that they may be in direct contact with the skin of the human user wearing the headgear article 200.

According to one example embodiment, and as illustrated in FIG. 2A, the neurofeedback headgear 200 includes a head receiving portion 216 and a brim portion 224 extending from the head receiving portion 216. The head receiving portion 216 refers to a portion of the headgear article 200 that receives the head of the human user and that is supported by the head when worn. The headgear article 200 illustrated in FIG. 2A is a baseball cap having a brim portion 224 extending from one portion of the edge of the headgear article 200. However, it will be understood that the example may be applied to any type of headgear article 200 having a brim portion 224, such as any hat having a partial brim (ex: visor, trucker hat, hardhat, baseball cap), or any hat having a full brim (ex: bucket hat, straw hat, cowboy hat). The brim portion 224 includes stiches 232 that maintain an external layer attached or affixed to the inner body of the brim portion. The stiches can also be used to affix an electrical connection module that connects the emitter 116 to the microcontroller (hidden under concealing layer 388), as will be explained in greater detail below.

In other examples, the receiving portion 216 of the headgear article 200 consists of a headband. According to such examples, the EEG sensor assembly 108 is integrated and concealed from view within the headband.

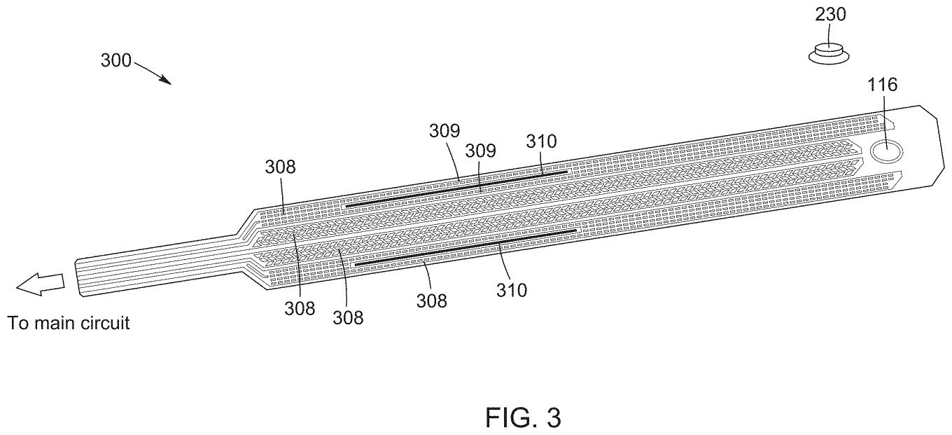

Continuing with FIG. 2A, the light emitting device 116 is mounted onto the neurofeedback headgear 200 such that it is located on a portion of the brim portion 224 that is visible to the user when the neurofeedback headgear 200 is worn by the user. For example, and as illustrated, the emitter 116 is located on an underside of the brim portion 224. So that the light emitting device 116 is located within the field of vision of the user, the light emitting device 116 may be located remotely of an edge of the head receiving portion 216, such as near an outer edge of the brim portion 224. In some example embodiments, a lens 230 (shown in FIG. 3) may be provided over the light emitting device 116 to focus the light emitted therefrom towards the eyes of the user.

According to one example embodiment, where the light emitting device 116 is located remotely of the edge of the head receiving portion 216 and the microcontroller 148 of the sensor assembly 108 is located on an interior surface of the head receiving portion 216, a signal path may be provided along a length of the brim portion 224 to connect the light emitting device 116 to the microcontroller 148. The signal path may be provided by a printed circuit board (PCB) module (not visible in FIG. 2A) extending along the length of the brim portion 224 with the light emitting device 116 being connected to the PCB module.

To conceal the PCB module of the brim portion 224, the PCB module may be disposed under an exterior layer of the brim portion 224. The exterior layer may be a fabric layer that is stitched to an inner body of the brim portion 224. To properly conceal the PCB module, the exterior layer is stitched to the inner body of the brim portion 224 after the PCB module has been placed against the inner body. During stitching, a stitching needle may contact or pierce through the PCB module.

Referring now to FIG. 3, therein illustrated is a perspective view of the PCB module 300 adapted for stitching according to one example embodiment. The stitching PCB module 300 includes a flexible substrate layer onto which are drawn a plurality of conductive traces 308. Each conductive trace 308 is formed of a plurality of conductive sub-traces having a grid-like arrangement so that small openings are defined within the grid arrangement. The grid-like arrangement of the sub-traces permit passage therethrough of a stitching needle during stitching while ensuring that the electrical paths defined by the conductive traces 308 remain intact. The PCB module 300 may further include a plurality of pre-formed holes 310 located adjacent to sides of the PCB module 300 to further reinforce the PCB module 300 against tearing during stitching. As illustrated, the pre-formed holes 310 extend adjacent to the sides along a portion of the length of the PCB module 300 intermediate the ends thereof.

It will be appreciated that a stitching needle piercing the PCB module 300 creates a tear in the PCB module 300. It was observed that the junction between a portion of the flexible substrate layer having a conductive trace and another portion of the flexible substrate layer that is free of the conductive trace, provides resistance against further tearing of the PCB module 300.

Furthermore, where the stitching needle pierces one of the branches of the grid-like arrangement of a sub-trace, another branch of the sub-traces continues to provide an electrical path to ensure the passage of signals.

It was observed that a stitching a needle contacting a longitudinal side of the PCB module 300 causes a strong torsional force to be applied on the PCB module 300, which increases the likelihood of tearing of the PCB module 300. It was further observed that the holes 310 extending adjacent to the sides of the PCB module 300 decreased the torsional force on any one location of the PCB module 300. The force caused by the stitching needle may be dispersed over portions of the PCB module 300 on either side of a pre-formed hole 310. The locations of the pre-formed holes 310 along the length of the PCB module 300 correspond to the locations of the stitches 232 that bond the exterior layer of the brim portion 224 to the inner body of brim portion 224.

The pre-formed holes 310 are located such that an electrically conductive portion 309 of a conductive trace 308 is located on either side of the holes 310. If one of the portions 309 is broken, such as being torn after being pierced by the stitching needle, the conductive trace 308 still provides a conductive path from the other conductive portions 309.

Referring back to FIG. 2A, according to one example embodiment, and as illustrated, the one or more sensing electrodes 124 a, 124 b of the EEG sensor assembly 108 are located on an interior sweat band 240 of the headgear article 200. The interior sweat band 240 refers to a lining that is provided near a bottom edge of the head-receiving portion 216 and which serves to capture sweat emitted from the user. The interior sweat band 240 will usually fit snugly against the forehead of the user wearing the headgear article 200.

The one or more sensing electrodes 124 a, 124 b include an electrically conductive layer that is disposed on an exposed surface of interior sweat band 240. The location of the sensing electrodes allows these to contact the skin of the user when the headgear article 200 is worn. In the illustrated example, the sensing electrodes 124 a, 124 b are placed at a frontal portion of the sweat band 240 so that the sensing electrodes 124 a, 124 b contact the forehead of the user when the headgear article 200 is worn.

In one example embodiment, each sensing electrode 124 a, 124 b may further include a resilient backing member that supports the conductive layer and biases the conductive layer towards the skin of the user when the headgear is worn by the user. The biasing ensures that a sufficient contact is made between the conductive layer of a sensing electrode and the skin of the user, so that electrophysiological signals of the brain are properly captured by the sensing electrode. For example, the resilient member may be a foam member. The foam member may be formed of silicon, EDPM rubber or neoprene.

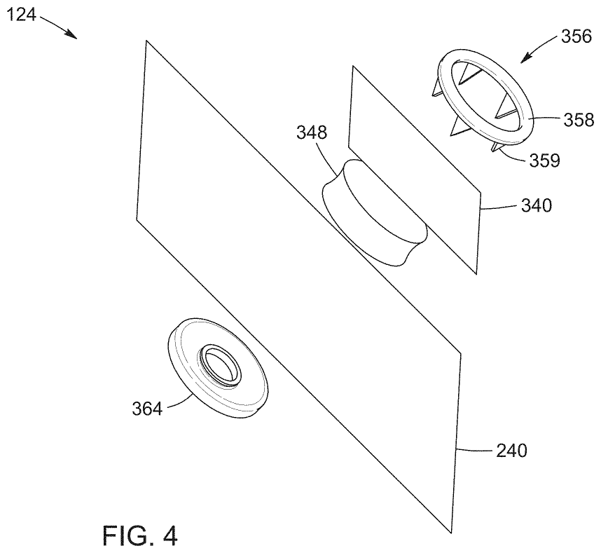

Referring now to FIGS. 4 to 6, therein illustrated are different views of a sensing electrode 124 according to one example embodiment. The conductive layer 340, the resilient member 348 and the sweat band 240, are positioned between cooperating snap rings 356 and 364. The resilient member 348 is further positioned between the conductive layer 340 and the sweat band 240. Of course, in other embodiments, the sweat band may be replaced with any support layer of a wearable device, and preferably a skin-contacting fabric. The cooperating snap rings 356 and 364 physically engage one another and retain together the conductive layer 340, the resilient member 348 and the sweat band or skin-contacting layer 240. When engaged, the exposed snap ring 356 rests on an exposed surface of the sweat band 240 and the hidden snap ring 364 is concealed between the sweat band/skin contacting layer 240 and the interior surface of the neurofeedback headgear 200.

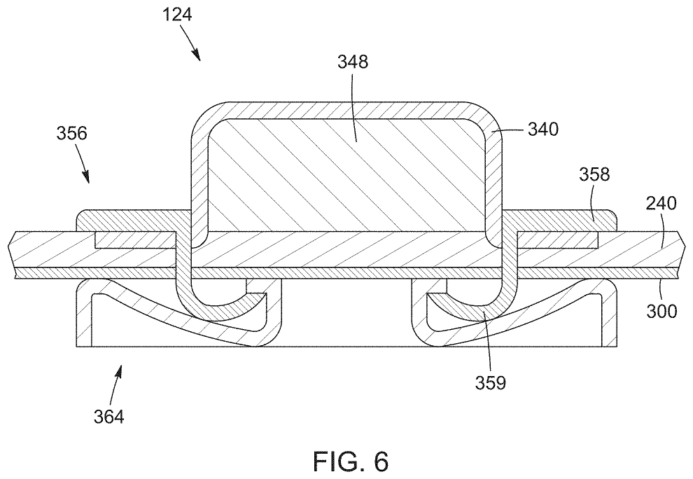

As best shown in FIG. 6, the exposed snap ring 356 is preferably an open ring 358 with prong/teeth members or spikes 359 that pierce the conductive layer 340 and the sweat band 240 and engage the other hidden snap ring 364 or socket, to hold the conductive layer 340 and resilient member 348, such as foam, in place over the sweat band or other skin contacting layer 240. Since the conductive layer 340 contacts the metallic rings, the flexible PCB stripes can be connected to the hidden snap ring, to conduct EEG signals captured by the conductive layer 340 to the signal receiving/conditioning module 132 and to the microcontroller 148. Preferably, as best shown if FIG. 6, a PCB module, preferably a flexible PCB strip 300′, is snapped between the snap rings 356, 364, with the traces 308 of the PCB strip 300′ contacting the metallic/conductive ring 364. Alternatively, the PCB strip 300′ can be connected to cooperating snap rings, with in this case a plug or stud protruding from one of the rings, the stud being snappable in the opening of ring 364.

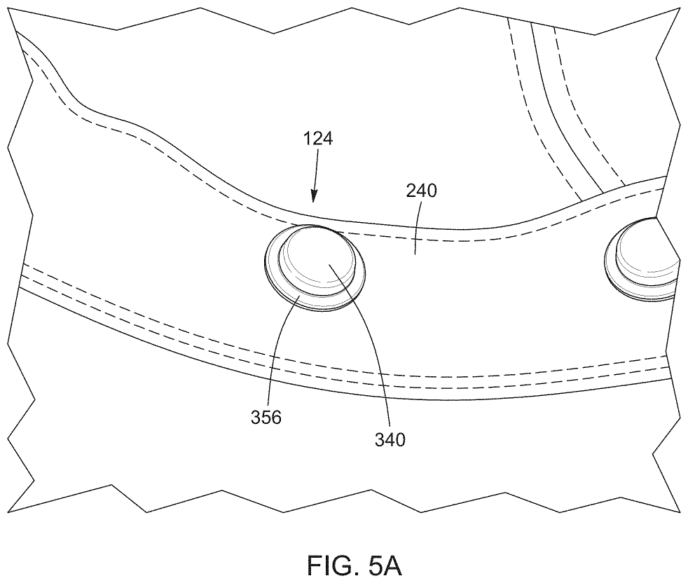

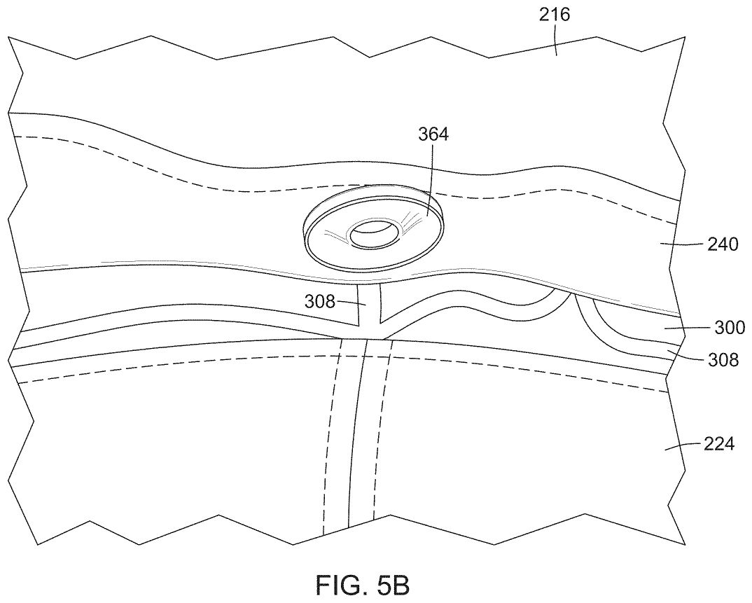

FIG. 5A illustrates a close-up view of an assembled EEG sensing electrode 124 attached to the inner sweat band 240 of the headgear article 200. As can be appreciated, the conductive fabric bulges outwardly from the skin contacting layer 240, providing increased contact surface with the forehead or skin of the user. FIG. 5A shows the skin contacting side of the sweatband or liner, while FIG. 5B illustrates the rear side of the sweat band or skin contacting layer, showing the rear/inner ring 364.

The EEG sensor assembly 108 mounted onto the headgear 200 may further include at least one additional flexible electrode PCB module, which may also be referred to as a PCB submodule, for connecting the at least one sensing electrode 124 to the signal receiving module 132. The additional PCB modules are similar to the one shown in FIG. 3. Where the EEG sensor assembly 108 includes a plurality of sensing electrodes 124 positioned along the sweat band 240 of the headgear article 200, the flexible PCB strip may extend along the sweat band 240 to interconnect the sensing electrodes 124 a, 124 b and to further connect the electrodes to the signal receiving module 132. Of course, in other embodiments, the connection between the sensing electrodes and the signal receiving module and/or the microcontroller can be made with other types of connections, such as with wires for example.

According to one example embodiment, portions of the flexible electrode PCB module are positioned between the snap rings 356, 364, such as between the lower/rear snap ring 364 and the sweat band 240. Accordingly, engagement of the snap rings 356, 364 causes the teeth members thereof to engage the flexible electrode PCB module, which holds the flexible electrode PCB module in place.

In other example embodiments, the signal receiving module 132 is positioned along an interior surface of the head-receiving portion 216 of the headgear article 200 and each sensing electrode 124 is connected to the signal receiving module 132 via a separate flexible electrode PCB that extends along the interior surface of the head-receiving portion 216.

In one example embodiment, the ground electrode 128 is located such that at least one sensing electrode 124 and the ground electrode 128 are located at substantially the same distance above an eye of the wearer when the wearable device 100 is worn. For example, the ground electrode 128 is also located on the interior sweat band 240 of the headgear article 200. It was observed that the blinking of the eyes of the wearer of causes a significant change in the signal being captured by a sensing electrode 124, which may skew the electroencephalographic signal being captured. The ground electrode 128 acts as a reference from the sensing electrodes 124, such as when the sensing electrodes 124 are connected to a differential amplifier. It was further observed that placement of the ground electrode 128 at the same distance above an eye of the wearer as at least one sensing electrode 124 causes a substantially equal change to the ground electrode 128 due to the blinking of the eyes of the wearer. Because the ground electrode 128 as a reference is changed by a substantially equal amount as the change to the sensing electrode, the change to the sensing electrode is substantially offset and the change due to blinking is not captured in a significant way.

According to one example embodiment, a first sensing electrode 124 b is placed along a first side of the interior sweat band 240 so as to be located above a left eye of the wearer when the wearable device 100 is worn. The ground electrode 128 is further placed along a second side of the interior sweat band 240 so as to be located above a right eye of the wearer when the wearable device 100 is worn. A second sensing electrode 124 a is centrally located between the first sensing electrode 124 b and the ground electrode 128. Accordingly, when a change is caused to the first sensing electrode 124 b due to blinking of the wearer, a substantially equal change is caused to the ground electrode 128.

Referring now to FIG. 2B, therein illustrated is a perspective view of an underside of a baseball cap 200′ according to a configuration that is well-known in the art. The baseball cap 200′ includes 6 panels 380 a, 380 b, 380 c, 380 d, 380 e, and 380 f that are pieced together to form the head receiving portion 216.

Referring back to FIG. 2A, where the EEG sensor assembly 108 is mounted onto a neurofeedback headgear 200 that is a baseball cap, a plurality of components of the EEG sensor assembly 108 are positioned on the two front panels 380 a, 380 b of baseball cap and a concealing panel 388 is disposed over the two front panels 380 a, 380 b to conceal these components. Only externally-interfacing components of the EEG sensor assembly 108 are left exposed, such as the sensing electrodes 124 a 124 b, the input/output port 160 and an on/off switch 396.

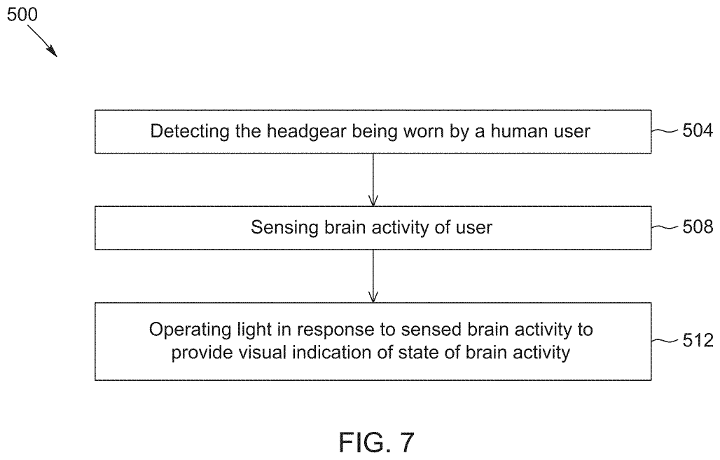

Referring now to FIG. 7, therein illustrated is a flowchart of the operative steps of a method 500 according to one example embodiment for sensing brain activity using the neurofeedback headgear 100 described herein. For example, the method may be carried out by the microcontroller 148 executing computer-readable instructions.

At step 504, whether or not the neurofeedback headgear is being worn by a human user is detected. In one example embodiment, whether the neurofeedback headgear is being worn, is detected automatically. The EEG sensing assembly 108 may be in a low-power idle mode when less than all of the sensing electrodes 124 a, 124 b, are in contact with a skin of a user. In the low-power idle mode, sensing of brain activity is not being carried out and only detecting of whether the headgear article 200 is being worn is carried out. Upon detecting that one or more of the electrodes 124, 128 are in contact with the skin of the user, the EEG sensing assembly 108 then enters into a sensing mode to sense brain activity.

At step 508, brain activity of the user is sensed. The sensing includes receiving electrophysiological signals captured by the one or more electrodes 124 a, 124 b and analysing the signals to determine a current state of brain activity.

At step 512, the emitter 116 is operated in response to the monitored brain activity to provide a visual indication of the state of brain activity to the user. The operation of the light emitting device 116 includes transmitting different control signals for controlling device 116 based on different current states of the brain activity.

The light emitting device 116 may be controlled in real-time to provide real-time visual feedback to the user. Accordingly, changing the visual indication emitted by the light emitting device 116, is used to indicate a change in a state of the brain activity. The sensing may be carried out continuously over an interval of time to monitor the brain activity of the user over that interval of time. The state of the brain activity may be a current concentration level of the user. For example, the light emitting device 116 may be controlled to emit different signals as the current concentration level of the user changes. The state of the brain activity may be a current meditation level of the user. For example, the light emitting device 116 may be controlled to emit different signals as the current meditation level of the user changes. The state of the brain activity may indicate the occurrence or the onset of a brain event. For example, the brain event may be the onset of an epileptic episode and the light emitting device 116 may be controlled to emit a particular visual feedback signal associated to such an event. The brain event may also be one or more of a change in state of relaxation, symmetry or asymmetry of brain activity, or onset of fatigue.

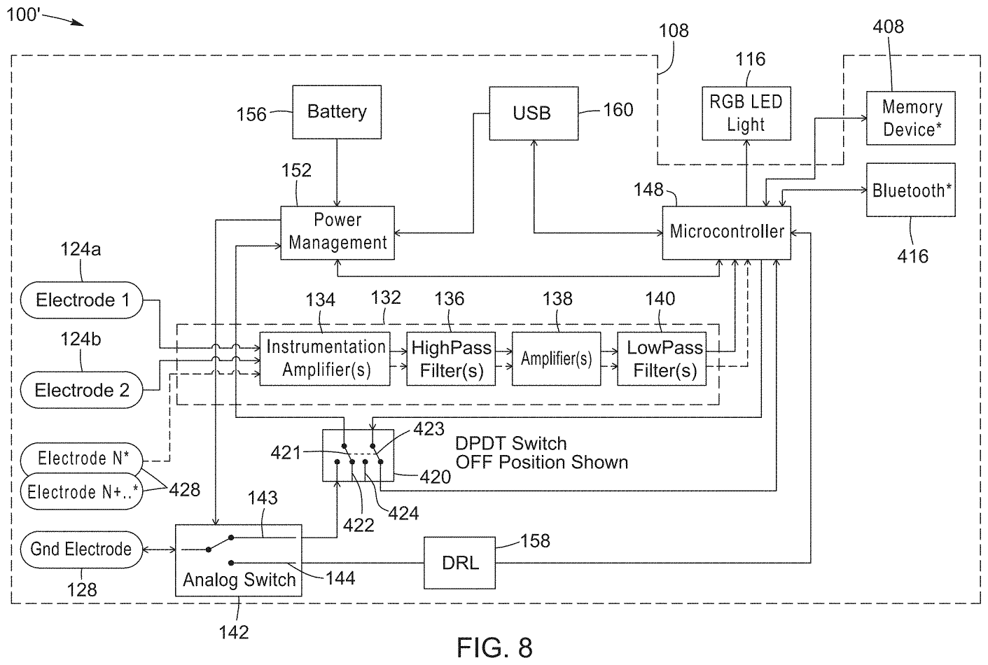

Referring now to FIG. 8, therein illustrated is an alternative EEG system 100′ according to an example embodiment. According to the alternative system 100′ a memory device 408 is provided to record sensed brain activity analyzed by the microcontroller 148. Furthermore, a wireless communication device 416 is provided to wirelessly transmit the sensed brain activity. The sensed brain activity may be transmitted in real-time to an external device having a display so that the sensed brain activity may be displayed in real time. The alternative system 100′ may further include additional sensing electrodes 428 to more accurately sense the brain activity of the user and/or to sense activity in different parts of the brain of the user.

Advantageously, various examples of embodiments described herein integrate an EEG sensing system within a wearable article. The sensing system is portable and wearable, which allows EEG signals to be sensed at all times and in various different situations of the daily life of the user. Furthermore, a light emitting device being located in the field of view of the user allows real-time feedback of the current brain activity of the user to be provided instantaneously to the user. Furthermore, by concealing the components of the EEG sensing system within the interior of the headgear article, the system may be worn discretely and without causing embarrassment to the user. The microcontroller of the EEG sensor assembly can be programmed to trigger the emitter not only based on a concentration level of the user, but also when a brain event is detected, such as for example the onset of a seizure or an epileptic crisis, a change in state of relaxation, symmetry of brain activity, and onset of fatigue.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the invention.

1. An electroencephalogram (EEG) sensing assembly, comprising:
a support layer having an outer side and an opposite inner side for facing a head of a user;
at least one sensing electrode, comprising:
an electrically conductive layer positionable against the inner side of the support layer;
a resilient member positionable between the support layer and the electrically conductive layer; and
a pair of cooperating snap rings, comprising:
a first snap ring positionable against the conductive layer, the first snap ring comprising prongs for engaging through the electrically conductive layer, the resilient member, and the support layer; and
a second snap ring positionable against the outer side of the support layer, the second snap ring comprising a socket for snap-fitting the prongs of the first snap ring thereby retaining together the electrically conductive layer, the resilient member and the support layer.

2. The EEG sensing assembly of claim 1, wherein the resilient backing member supports the conductive layer and biases the conductive layer towards the skin of the user through an opening of the first snap ring when the first snap ring engages the second snap ring.

3. The EEG sensing assembly of claim 2, wherein the prongs of the first snap ring extend inwardly to engage the socket when the first snap ring is snap-fitted to the second snap ring.

4. The EEG sensing assembly of claim 3, wherein a first section of the socket of the second snap ring extends upwardly and a second section of the socket projects radially to snap-fit the prongs of the first snap ring.

5. The EEG sensing assembly of claim 4, wherein each of the first snap ring and the second snap ring comprise a metallic material.

6. The EEG sensing assembly of claim 4, further comprising a printed circuit board (PCB) layer for operatively connecting the at least one sensing electrode to a microcontroller.

7. The EEG sensing assembly of claim 6, wherein at least a portion of the PCB layer is positionable between the first snap ring and the second snap ring.

8. The EEG sensing assembly of claim 7, wherein at least a portion of the PCB layer is electrically connectable to the second snap ring.

9. The EEG sensing assembly of claim 8, further comprising a stud snappable in an opening of the second snap ring for electrically connecting to the PCB layer.

10. The EEG sensing assembly of claim 6, wherein the PCB layer comprises a flexible substrate layer and a plurality of conductive traces drawn thereon.

11. The EEG sensing assembly of claim 10, each of the plurality of conductive traces comprises a respective plurality of conductive sub-traces having a respective grid-like arrangement defining a respective opening.

12. The EEG sensing assembly of claim 10, wherein the PCB layer further defines a plurality of longitudinally extending slots adjacent lateral sides thereof for reinforcing the PCB layer.

13. The EEG sensing assembly of claim 6, wherein the resilient backing member comprises a foam member.

14. The EEG sensing assembly of claim 7, wherein the foam member comprises at least one of silicon, ethylene propylene diene monomer rubber (EDPM) rubber, and neoprene.

15. The EGG sensing assembly of claim 1, wherein the EEG sensing assembly is concealable within a headband to be worn on the head of the user.

16. The EEG sensing assembly of claim 1, further comprising:
a microcontroller operatively connectable to the at least one sensing electrode.

17. The EEG sensing assembly of claim 16, further comprising:
a power management module operatively connectable to the microcontroller, the power management module comprising a power management circuit operatively connected to a battery.

18. The EEG sensing assembly of claim 17, further comprising:
a ground electrode positioned adjacent to the at least one sensing electrode such that the ground electrode and the at least one sensing electrode are located at a same distance above an eye of the user.

19. An electroencephalogram (EEG) sensing assembly, comprising:
a support layer having an outer side and an opposite inner side for facing a head of a user;
at least one sensing electrode, comprising:
an electrically conductive layer positioned against the inner side of the support layer;
a resilient member positioned between the support layer and the electrically conductive layer; and
a pair of cooperating snap rings, comprising:
a first snap ring positioned against the conductive layer, the first snap ring comprising prongs for engaging through the electrically conductive layer, the resilient member, and the support layer; and
a second snap ring positioned against the outer side of the support layer, the second snap ring comprising a socket for snap-fitting the prongs of the first snap ring thereby retaining together the electrically conductive layer, the resilient member and the support layer.

20. The EEG sensing assembly of claim 19, further comprising:
a printed circuit board (PCB) layer for operatively connecting the at least one sensing electrode to a microcontroller.
